(19) United States
(12) Patent Application Publication
(10) Pub. No.: US 2015/0216051 A1
(43) Pub. Date: Jul. 30, 2015

(54) METHOD OF FABRICATING ELECTRICAL FEEDTHROUGHS USING EXTRUDED METAL VIAS

(71) Applicants: Kedar G. Shah, San Francisco, CA (US); Satinderpall S. Pannu, Pleasanton, CA (US)

(72) Inventors: Kedar G. Shah, San Francisco, CA (US); Satinderpall S. Pannu, Pleasanton, CA (US)

(73) Assignee: LAWRENCE LIVERMORE NATIONAL SECURITY, LLC, LIVERMORE, CA (US)

(21) Appl. No.: 14/367,759

(22) PCT Filed: Dec. 21, 2012
(86) PCT No.: PCT/US12/71392

§ 371 Date: Jun. 20, 2014

Related U.S. Application Data
(60) Provisional application No. 61/578,806, filed on Dec. 21, 2011.

(51) Int. Cl. H05K 1/11; H05K 3/14

(52) U.S. Cl. CPC H05K 1/115; H05K 3/14; H05K 2201/09827

(57) ABSTRACT

A method of fabricating high-density, preferably bio-compatible, electrical feedthrough structures and interfaces by extruding electrically conductive material into electrically conductive film-coated throughholes formed on an electrically non-conductive substrate to form extrusion-formed electrically conductive vias which pass through the substrate for microelectronic applications.

### CROSS-REFERENCE TO RELATED APPLICATIONS

This patent document claims the benefit and priority of U.S. Provisional Application No. 61/578,806, filed on Dec. 21, 2011, hereby incorporated by reference.

### FEDERALLY SPONSORED RESEARCH OR DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### TECHNICAL FIELD

This patent document relates to methods of fabricating electrical feedthroughs, and in particular to a method of fabricating high-density electrical feedthroughs using extruded metal vias.

### BACKGROUND

Electrically-active bio-medical devices (such as for example pacemakers, cochlear implants, and neural prosthetics) have the ability to diagnose, monitor, and treat a wide range of diseases and conditions. In many of these applications, it is necessary to chronically implant the electronics that enable interactions with the tissue using electrical signals. However, because many of the component materials used in such devices are not bio-compatible, that is, they are toxic to the body and can induce undesirable biological reactions, it is critical to hermetically seal the non-bio-compatible components (e.g. integrated circuits, passive components, batteries) in a bio-compatible material, so that the body does not have a cyto-toxic response. Hermetic sealing also helps protects electrical components from damage due to moisture and the corrosive environment in the body. FIG. 1 shows a schematic illustration of a common hermetic encapsulation approach for implantable electronic devices, such as 10, where non-bio-compatible components and materials 11, such as electronics, are encapsulated in a hermetically sealed package 12 made of bio-compatible materials. In this arrangement, an array of hermetic electrically conducting feedthroughs 13 is provided on an electrically insulating portion 14 of the package 12 for use as electrical conduits which allow the transmission of electrical signals between the exterior and the interior of the package (e.g. between the body and the electronics housed within the package), while maintaining a seal that prevents transfer of particles or fluids. It is desirable that the feedthroughs be fabricated from chronically bio-compatible materials, while meeting stringent specifications for hermeticity.

Various methods are known to produce hermetic electrical feedthroughs. However, they often tend to be high-cost, lack scalability, and have inherent material incompatibilities Importantly, limited density of electrical feedthroughs, due to pitch limits affecting spacing between feedthroughs, can directly affect the performance of a bio-medical device, such as cochlear implants with limited fidelity, or retinal prostheses with limited image resolution. For example, one common method of making hermetic feedthroughs is by brazing metal pins inside the vias of an insulating substrate. While this method can consistently result in hermetic feedthroughs, the pitch is limited, in many cases to as high as 400-500 μm.

Another example approach is shown in FIGS. 2A and 2B illustrating a method for producing metal feedthroughs in laser drilled holes on non-conductive substrates. In this method, a ceramic or other electrically non-conductive substrate 20 is laser drilled with holes 21. The holes are turned into feedthroughs by filling them (e.g. by stencil printing) with thick-film metal paste 22 which consists of metal particles in an organic solvent. The metal paste is typically pulled through the holes using vacuum and co-fired at high temperature to drive out the solvent, leaving only metal in the holes. This method however can be problematic because the thick-film metal paste can leave voids when fired, or the adhesion of metal to substrate may be poor, either of which can cause leakage paths through the feedthroughs leading to hermetic failure. Voids in particular can be produced when the organic binders or thinners used in the metal paste are driven out during the firing process, and which may negatively impact the hermeticity of the feedthroughs. Also the high-temperature firing can cause delamination of the metal from the ceramic due to the stresses induced from thermal expansion mismatch between the metal and the ceramic. And because hermetic package enclosures (e.g. 12 in FIG. 1) are typically made of bio-compatible metals which must be hermetically bonded to ceramic feedthrough substrates (e.g. 14 in FIG. 1) using a high temperature brazing process, this introduces an additional high temperature process which can further increase the chances of failure at the feedthrough-ceramic and also the ceramic-package interfaces. And the laser cutting process used to form holes introduces additional limitations. For example, laser cutting often causes micro-cracks in the ceramic substrate, making it fragile and limiting the minimum gap between adjacent holes.

Also the minimum diameter of the substrate holes is restricted due to tapering produced by the laser cutting process which limits feedthrough density. As illustrated in FIGS. 3A and 3B, there exists a trade-off between substrate thickness (scalability) and hermeticity. Shorter holes (in which shorter feedthroughs 26 are formed) in thinner ceramic substrate 25 of FIG. 3B, are easier to laser cut, but they are less likely to be hermetic since there is a smaller area for the metal to adhere to the ceramic. Thicker ceramic substrates, such as 23 in FIG. 3A, provide more surface area for the metal to adhere and improve hermeticity. However, they are harder to laser cut, and as can be seen by the four longer feedthroughs 24 in FIG. 3A in the same substrate area as six shorter feedthroughs 26 in FIG. 3B, feedthrough density is less than a thinner substrate due to hole taper. Commercially available feedthroughs using this technology for bio-medical applications have a pitch in the range of 400-600 μM.

And another known method of producing hermetic electrically conducting feedthroughs uses low temperature co-fired ceramics (LTCC), and illustrated in FIGS. 4A-4D. In this method, multiple layers of thin ceramics 30-33 are physically punched or laser-machined with holes 34-37, respectively. Each ceramic layer 30-33 is then rnetalized using thick-film metal paste, 38-41, respectively, to fill the holes and create the feedthroughs. As shown in FIG. 4D, the layers of ceramics 30-33 are then aligned/stacked and sintered to create the final substrate with feedthroughs 43 extending through the stack. However, the size of holes formed using this method is often restricted to the dimension of the punching process (e.g. about 100-125 microns), which limits scalability. And the mechanical fragility of substrates due to punching can restrict the pitch between adjacent holes. Misalignment of individual feedthroughs due to uneven shrinkage of the ceramic during the sintering process is also possible and can cause mismatching between feedthrough locations and bond pads formed to connect to electronic components.

In order to improve the longevity and effectiveness of implantable devices, it is advantageous to be able to fabricate durable hermetic electrically conductive feedthroughs which allow connection to hermetically sealed electronic devices. In particular, it would be advantageous to provide a scalable fabrication method for producing high-density, bio-compatible, hermetic electrically conductive feedthroughs in a range of substrate thicknesses that improves the hermetic bond between feedthrough and insulator by using lower temperature process for insulator sealing. Longevity is an important consideration for implantable devices, such as for example cochlear implants, where it is desired that the implant continue to function reliably over the patient's entire lifetime without causing any adverse cytotoxic reaction from the tissue or having to be removed/replaced due to failure. And in the case of retinal prosthetics, for example, the number of electrical feedthroughs or channels may directly affect the image quality that can be restored to the patient. Simply increasing the number of feedthroughs is typically not feasible because it increases the size of implant, which may make it impractical for implantation. It is therefore necessary to increase the density of electrical feedthroughs so that the channel count can be increased without significantly affecting device size. Thus, there is a need for high-density, bio-compatible electrical feedthrough arrays.

### SUMMARY

The technology described in this patent document includes devices, systems and methods for fabricating high-density hermetic electrical feedthroughs, and the feedthrough interfaces, structures, and devices produced thereby.

In one example implementation, a method of fabricating an electrical feedthrough structure is provided comprising: forming at least one throughhole(s) from one side of an electrically non-conductive substrate to an opposite side of the substrate; conformally coating the substrate and the throughhole(s) with an electrically conductive film; and extruding an electrically conductive material into the film-coated throughhole(s) to form extruded electrical feedthrough(s) therefrom.

In another example implementation, an electrical feedthrough structure is provided comprising: an electrically non-conductive substrate having at least one throughhole(s) that is conformally coated with an electrically conductive film, and a corresponding number of electrical feedthroughs extrusion-formed in the film-coated throughhole(s) so as to be bonded to the throughhole walls.

These and other implementations and various features and operations are described in greater detail in the drawings, the description and the claims.

The present invention is generally directed to a method of fabricating high-density, preferably bio-compatible, electrical feedthroughs by extruding electrically conductive material into electrically conductive film-coated throughholes formed on an electrically non-conductive, i.e. insulating, substrate to form extrusion-formed electrically conductive vias which pass through (preferably hermetically) the substrate for microelectronic applications, as well as to the extrusion-formed electrical feedthrough structures themselves.

Various substrate materials may be used such as but not limited to alumina, zirconia, titania, glass, sapphire, silicon, etc. And the substrate thickness may also vary. For example, one example range may be from 10-10,000 microns. The substrate length and width are only relevant to the final application, and can cover the range from, for example, a few microns to a few feet. It is notable that alumina is a good choice for the insulating substrate because of its demonstrated bio-compatibility, chemical inertness, and use in bio-medical devices, such as retinal prostheses, cochlear implants, and neural stimulators. Example constructions of the present invention have used alumina substrates with a thickness of 250 μm and a surface roughness of 25-50 nm Ra.

Single or multiple throughhole(s) (i.e. at least one throughhole) are made in the substrate, either in a repeating array pattern or a random pattern, using various hole forming methods, such as but not limited to laser cutting, laser machining, mechanical drilling, reactive-ion etching, ion-milling, deep reactive ion etching, water-jet cutting, or wet etching, etc. The throughholes can have various diameter size ranges, such as for example a diameter range from 5-1000 microns. It is also appreciated that the throughholes may be lithographically defined, and can be squares/rectangles/other irregular shapes. Formation of throughholes by lithographic etching (using lithographically etchable insulating substrates) can further reduce scale/miniaturize the array of feedthroughs with ultra-high channel counts. Example constructions of the present invention have used laser processing technology to form throughholes at a pitch of 200 μm (equivalent to a density of −2500 vias/cm²) and having an inherent taper that results in the throughholes on the laser entry-side to be larger in diameter than the exit-side. The average exit-side via diameter for the samples was 53 μm (standard deviation 2.7)

The substrate is then conformally coated on both sides with an electrically conductive material, e.g. a metal thin film such as for example gold or titanium, including on the throughhole walls. The electrically conductive film may be, for example, a single or multi-layer stack (e.g. Ti/Au) deposited using, but not limited to physical vapor deposition, chemical vapor deposition, electroplating, sputtering, or atomic layer deposition, etc. And it is appreciated that the electrically conductive film may or may not be the same as the extruded metal via. It is also appreciated that an adhesion layer may also be deposited on the electrically conductive film material prior to extruding the electrically conductive material into the throughholes, to promote adhesion between the extruded electrically conductive material and the electrically conductive film. Or an adhesion layer may be deposited on the substrate and the throughholes (i.e. on the throughhole walls), prior to depositing the electrically conductive film, to promote adhesion between the electrically conductive film and the substrate and the thorughhole. The adhesion layer material may be selected from a type known to have good adhesion with both the electrically conductive film and the extruded via material, while also being a good electrical conductor. Example materials for the adhesion layer may include, for example, titanium (good adhesion to gold and alumina) or another metal, self-assembled monolayers, or other adhesion promoters. And example deposition methods of the adhesion layer include, for example, physical vapor deposition (sputter or e-beam), chemical vapor deposition, (atomic layer deposition, plasma enhanced chemical vapor deposition, etc.), electrochemical deposition, vapor deposition, etc.

The electrically conductive film (i.e. the metalized film) may then be patterned to form discrete electrically conductive forms e.g. metal traces, for connecting to electronic chips or passive electrical components on either side of the substrate. It is appreciated that patterning may occur prior to the extrusion step, or after the extrusion step, e.g. after the polishing step shown in FIG. 6E.

Next, an electrically conductive material (e.g. metal) is extruded into the throughhole using, for example, a stud bumper or flip-chip bonder, and any combination of elevated temperature, applied force, and ultrasonic energy, to shear the material (of a larger size than the throughhole) causing it to deform and fill the throughhole and produce a strong diffusion bond with the throughhole walls, and ultimately form the extrusion-formed electrical feedthroughs. And a seal is formed along the wall of the via when a stud bump of larger diameter than the via is extruded through it. Two example extrusion methods include direct stud-bumping stud bumps into the througholes, and flip-chip thermocompression to transfer metal formed on a sacrificial substrate into the throughholes of the electrically non-conductive substrate. The parameters which may be used in the extrusion process include ultrasonic bonding power, time that ultrasonic energy is applied (or ultrasonic time), bond force, substrate temperature, and wire hardness. Example constructions of the present invention have used commercially available 25 μm diameter gold bonding wire of high and low hardness was utilized, and stud bumps were formed with an F&K Delvotec 5610 bonder. Many such feedthroughs can be created on the same substrate to result in a high-density array of hermetic feedthroughs. The material extruded through the vias may be any electrical conductor (such as but not limited to gold, platinum, aluminum, copper, rhodium, ruthenium, palladium, niobium, titanium, iridium and their alloys). In some embodiments, the electrical feedthroughs that are formed may have a rivet-like shape comprising a shank portion positioned in the throughhole, and a head portion positioned against an outer surface of the substrate at an inlet end of the throughhole.

After the extrusion step, various additional steps may be employed, such as for example, electroplating additional metal to fill the feedthroughs, or high temperature annealing the extruded metal. The substrate may also be polished flat and may be metalized and patterned for final application. In one embodiment, the grinding/polishing step is used to electrically separate the feedthroughs from each other by removing the conformally coated electrically conductive film everyone from the substrate except on the throughhole walls. In another embodiment, a compressive force may be applied to the extruded stud bumps to drive the extruded stud bumps further into the throughbores. In particular, the head portions of the extruded stud bumps may be coined by thermo-compression on a flip-chip bonder. This process serves to make the head portion of the extruded vias planar, but it also can improve the hermeticity of feedthroughs.

The method of the present invention would provide various benefits. It does not use filler/binder material in the via material which can result in a less porous material with a higher hermeticity. And smaller vias are possible than stencil printing due to definition of metal using lithography or stud-bumping, which can produce higher density via arrays. In addition, the extruded feedthrough process can reduce the required thickness of the substrate, by enabling hermetic feedthroughs at roughly half the thickness of conventional feedthrough technologies because it uses bulk metal wire to seal the via openings. And in addition to the high density, the approach of the present invention can be used to form hermetic feedthroughs at extremely low processing temperatures (e.g. 150° C.), and has the ability to rapidly create feedthroughs without complicated processing steps. The extruded via process of the present invention would also enable an assembly process in which all electronic components may be assembled first and the via array produced last. This would reduce the chance of failure of the feedthrough array, and also make it easier to perform electrical testing of the assembled components before they are hermetically sealed.

As previously discussed, hermetically sealed packages with electrical feedthroughs are commonly used by many companies in the bio-medical device industry to separate non-bio-compatible components from bodily tissue. However, electrical feedthroughs are also heavily used in the semiconductor industry to interconnect electronic chips. As such, the extruded electrical feedthrough structures and method of fabrication of the present invention may also be used in non-bio-medical applications, such as separating sensors or electronics from harsh environments in the field. It is appreciated therefore that while bio-compatible materials are preferred for use as the extruded electrically conductive feedthroughs of the present invention when used in bio-medical implant applications, other non-bio-compatible materials may be used in the alternative for other non-bio-medical applications. Also, while hermetic feedthroughs are critical in bio-medical applications, non-hermetic extruded feedthrough structures may also be fabricated according to the present invention for other types of applications, such as known in the microelectronics industries. The challenge in all these applications, however, remains the same, that is to create very high-density electrical feedthroughs using materials that are compatible with the environment of application.

### BRIEF DESCRIPTION OF THE DRAWINGS

FIG. 1 is a schematic view of an implantable device illustrating a common approach to encapsulating non-bio-compatible component materials in a bio-compatible sealed package.

FIG. 2A is a cross-sectional view of a substrate with holes produced by laser cutting in a first example method of fabricating feedthroughs known in the art.

FIG. 2B is a cross-sectional view of the substrate in FIG. 2A after the laser-cut holes are filled with a metal from a metal paste.

FIG. 3A is a cross-sectional view of an example thicker substrate produced by the method illustrated in FIGS. 2A-B illustrating, together with FIG. 3B the trade-off between substrate thickness (scalability) and hermeticity.

FIG. 3B is a cross-sectional view of an example thinner substrate produced by the method illustrated in FIGS. 2A-B illustrating, together with FIG. 3A the trade-off between substrate thickness (scalability) and hermeticity.

FIGS. 4A-D show four stages of a second example method of fabricating feedthroughs known in the art by co-firing multiple ceramic substrates.

FIGS. 5A-F show six stages of an example method of fabricating an hermetic electrical feedthrough device of the present invention using extruded electrically conductive material.

FIGS. 6A-E show five stages of another example method of fabricating an hermetic electrical feedthrough device of the present invention using extruded electrically conductive material.

FIGS. 7A-E show five stages of another example method of fabricating an hermetic electrical feedthrough device of the present invention using extruded electrically conductive material.

FIG. 8 shows an example electrical feedthrough device having stepped throughholes and extrusion-formed electrical feedthroughs therein.

FIG. 9 shows an example electrical feedthrough device having tapered throughholes and extrusion-formed electrical feedthroughs therein.

FIG. 10 shows a photo of an example substrate with formed throughholes.

FIG. 11 shows a top view photo of an extrusion-formed electrical feedthrough after coining.

FIG. 12 shows a cross-sectional view photo of the coined extrusion-formed electrical feedthrough of FIG. 11.

FIG. 13 shows an enlarged top view photo of an uncoined extrusion-formed electrical feedthrough.

FIG. 14 shows an enlarged cross-sectional view photo of the uncoined extrusion-formed electrical feedthrough of FIG. 13.

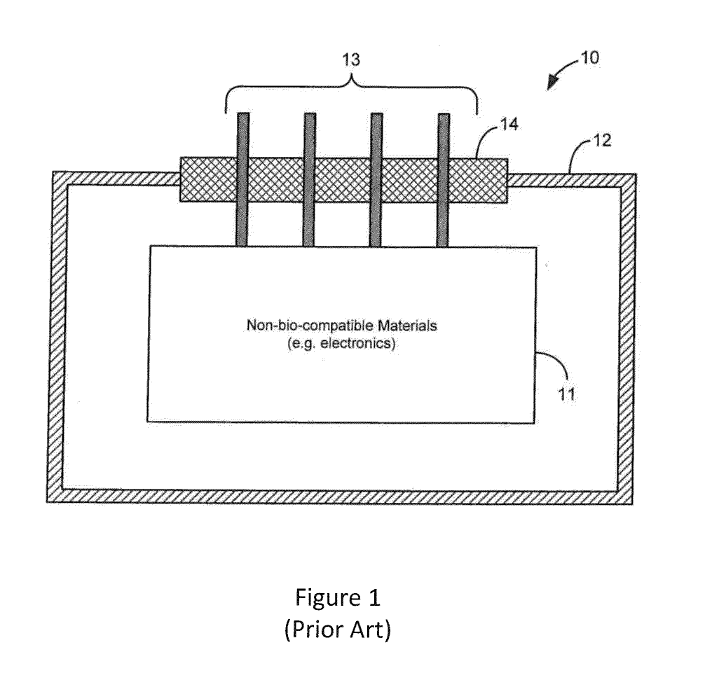

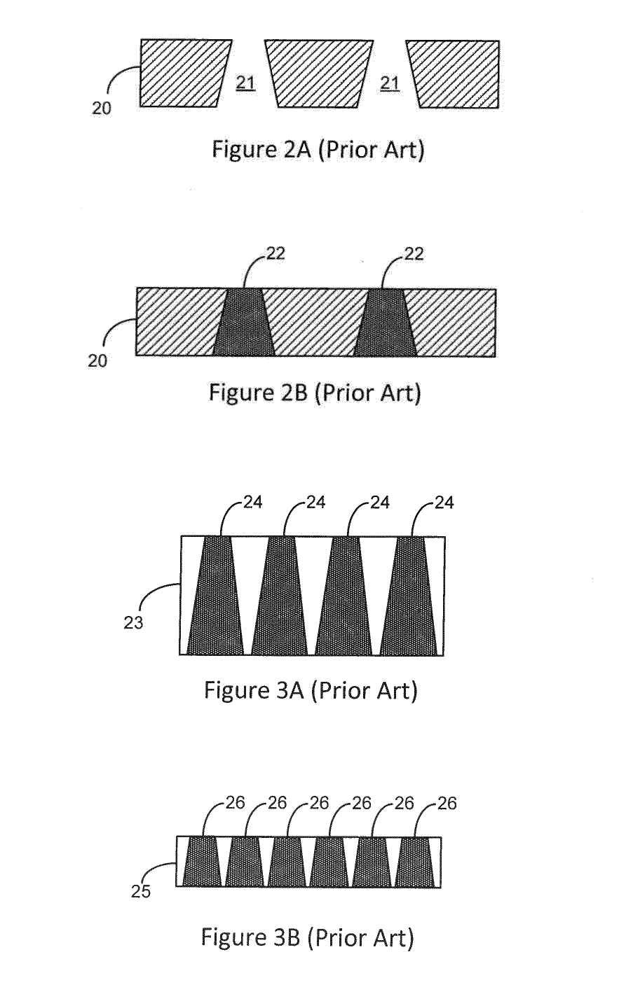

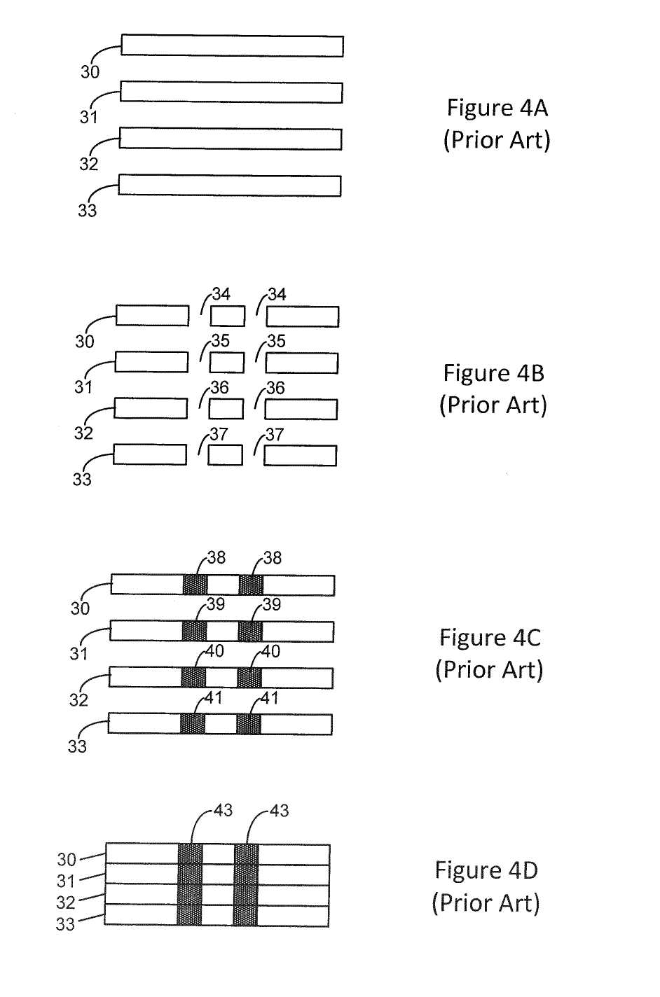

### DETAILED DESCRIPTION

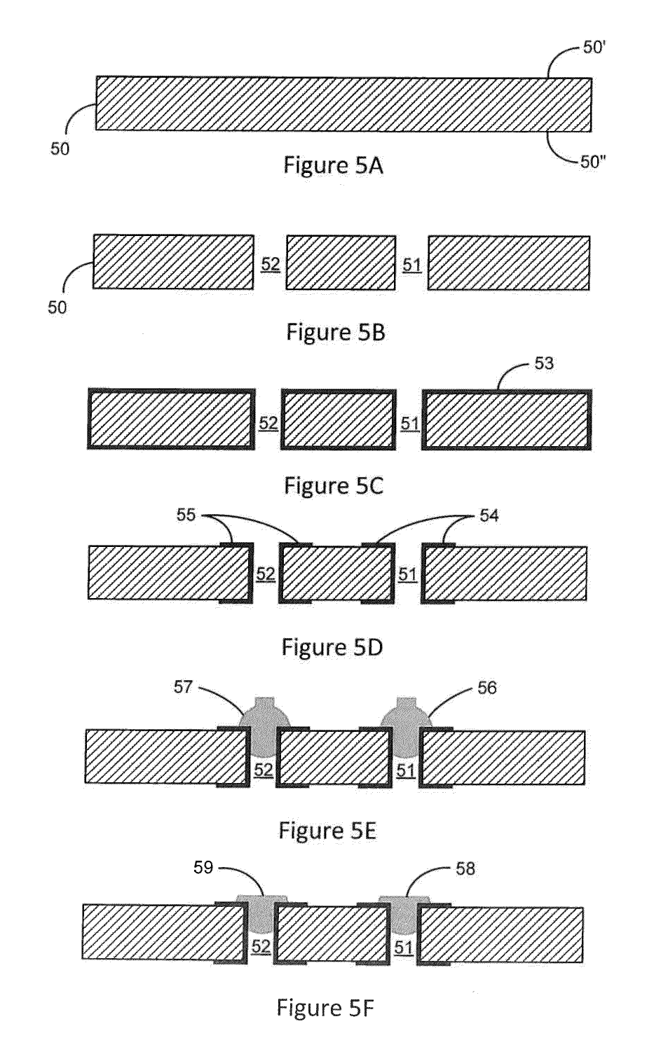

Turning now to the drawings, FIGS. 5A-F shows one example embodiment of the fabrication method of the present invention using direct stud-bumping of stud bumps into substrate throughholes to extrude the stud bumps into the throughholes.

First, as can be seen in FIG. 5A an electrically non-conductive substrate 50 is provided having opposing sides 50′ and 50″. And in FIG. 5B representative throughholes 51 and 52 are shown created in the substrate between the opposing sides, by various methods as discussed in the Summary. It is appreciated that only a single throughhole may be formed, or in the alternative, additional throughholes may be formed to produce an array of densely-packed throughholes. In any case, the throughholes may also characterized as via holes or via openings, through the substrate. In FIG. 5C the substrate is conformally coated on both sides with an electrically conductive thin film 53. Notably, the conductive thin film is also coated in the throughholes on the throughhole walls.

In FIG. 5D the thin film is next shown patterned (e.g. lithographically) on both sides of the substrate to form discrete electrically-conductive forms 54 and 55 which are electrically separated from each other. In particular, the electrical forms 54 and 55 are shown centered about the throughholes 51 and 52, and include the throughhole wall coatings. It is appreciated also that the electrically conductive forms formed by patterning may also include metal traces connecting the metallized throughholes with other regions on the substrate.

In FIG. 5E representative stud bumps 56 and 57 are shown formed and extruded partially in the throughholes 51 and 52, respectively, to form extruded electrically conductive feedthroughs or vias. The stud bumps may be formed and extruded using a wire-bonder or stud-bumper, which applies at least one of force (0.1-10000N per extrusion), temperature (room-temperature to 1000 C), and ultrasonic energy. Furthermore, the stud bumps are formed, for example, with diameters that are larger than the corresponding throughhole such that extrusion into the throughholes may take place while bonding to the metallized substrate. In manner of using extrusion, ultrasonic energy, elevated temperature, and force, a hermetic seal is produced between the extrusion-formed feedthroughs/vias and the substrate. Simultaneously, the diffusion of the stud bump into the conformal metallization provides an electrically conductive path between the two surfaces of the alumina substrate.

And FIG. 5F shows the electrical feedthroughs 58 and 59 after a compressive force (e.g. thereto-compressive force) is applied to the bumps 56 and 57, such as in a coining process, to further to drive the bumps deeper into the throughholes, and to shape the outer surface (e.g. flatten into a flat surface) onto which electronic components may be assembled. It can be seen that the electrical feedthroughs thus formed may each having a rivet-like shape comprising a shank portion positioned in the throughhole, and a head portion positioned against the substrate surface at an inlet end of the throughhole.

FIG. 10 shows a photo of an example array of twelve laser-machined via holes (throughholes) 100 (200 μm pitch) in a metallized ceramic substrate, and FIG. 11 shows a photo of the extruded vias 101 formed in the via holes from gold stud bumps.

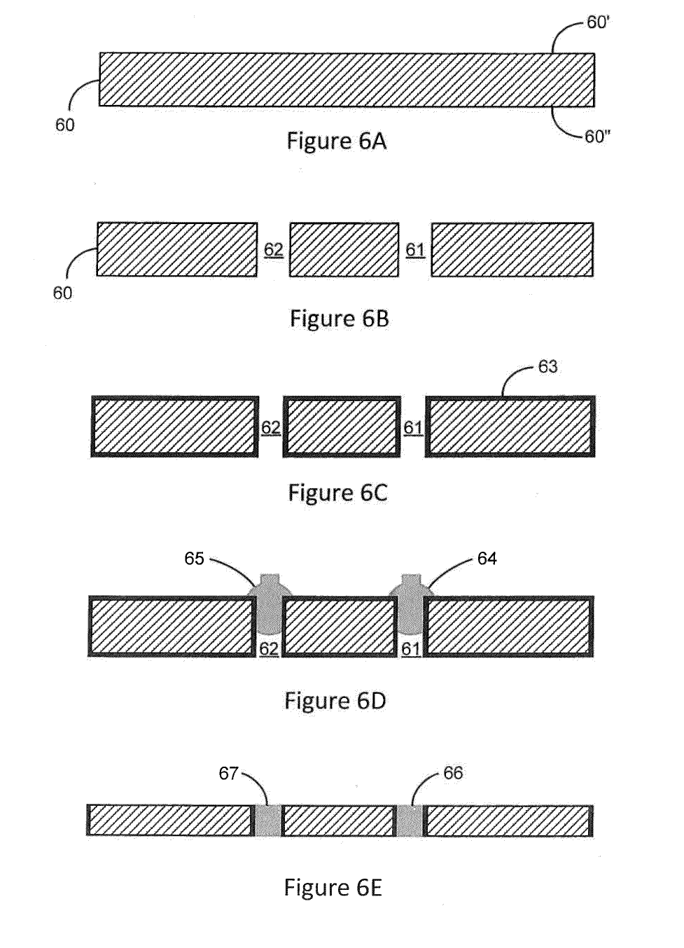

FIGS. 6A-E show another example embodiment of the fabrication method of the present invention using direct stud-bumping to extrude stud bumps into the throughholes. In this case, FIGS. 6A-C are similar to FIGS. 5A-C in that a substrate 60 is provided having opposing surfaces 60′ and 60″, throughholes 61 and 62 are formed through the substrate, and the substrate is conformally coated with an electrically conductive film coating.

In FIG. 6D, however, stud bumps 64 and 65 are shown formed and extruded into the film-coated throughholes 61 and 61, respectively, without first patterning the electrically conductive coating into discrete electrical forms. And in FIG. 6E, the substrate and extrusion-formed bumps are ground and polished on both sides to remove the electrically conductive coating from the opposing sides of the substrate and Ruin electrically separated feedthroughs/vias 66 and 67.

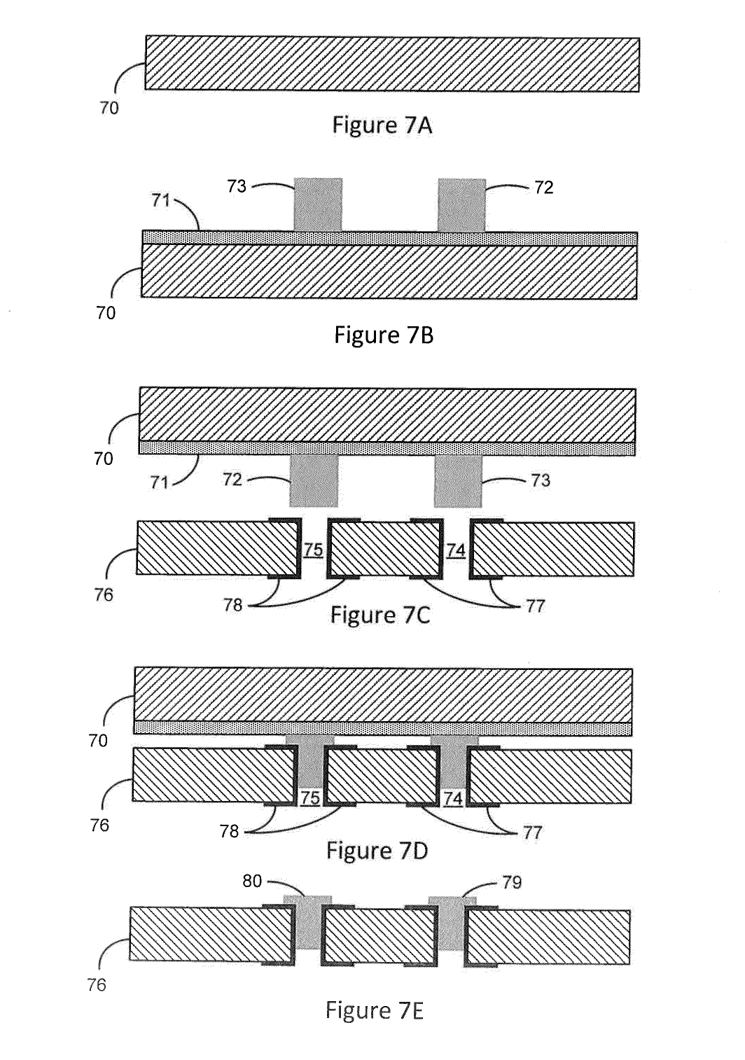

FIGS. 7A-E show another example embodiment of the fabrication method of the present invention using flip-chip thermo-compression to extrude stud bumps to the throughholes by transferring electrically conductive material from a sacrificial substrate. In FIGS. 7A-B, a sacrificial substrate 70 is shown provided, upon which representative electrically conductive posts 72 and 73 are created, such as for example by stud-bumping or electroplating. The posts on the sacrificial substrate may share a similar pattern and shape as the throughholes in the feedthrough substrate. Here too the post size/diameter is larger than the throughhole through which the post is extruded. And the sacrificial substrate can consist of a single material or a material with a lift-off layer 71, such as shown in FIG. 7B, for removal of the sacrificial substrate from the electrically conductive post after the extrusion step shown in FIG. 7D.

In FIG. 7C, a tool such as a flip-chip bonder (not shown) is used to flip the sacrificial substrate 70 and align the posts 72 and 73 formed thereon to corresponding throughholes, 75 and 74, formed on a non-conductive substrate 76 having patterned electrically conductive forms 77 and 78, similar to FIG. 5D. It is notable that instead of the substrate 76 similar to FIG. 5D, a non-patterned, conformally coated substrate such as shown in FIG. 6C may be used.

And as shown in FIG. 7D, the posts 72 and 73 are extruded into corresponding throughholes 75 and 74 by any combination of force, elevated temperatures, and ultrasonic energy, such as may be provided by the flip-chip bonder tool. And in FIG. 7E, the sacrificial substrate 70 is removed by etching it away or using the lift-off layer 71 in a lift-off process to separate it from the extrusion-formed electrical feedthroughs 79 and 80. It is appreciated that if a non-patterned, conformally-coated substrate such as shown in FIG. 6C was used, then the final step would involve grinding and polishing on both sides to remove the electrically conductive coating from the opposing sides of the substrate and form the electrically separated feedthroughs/vias.

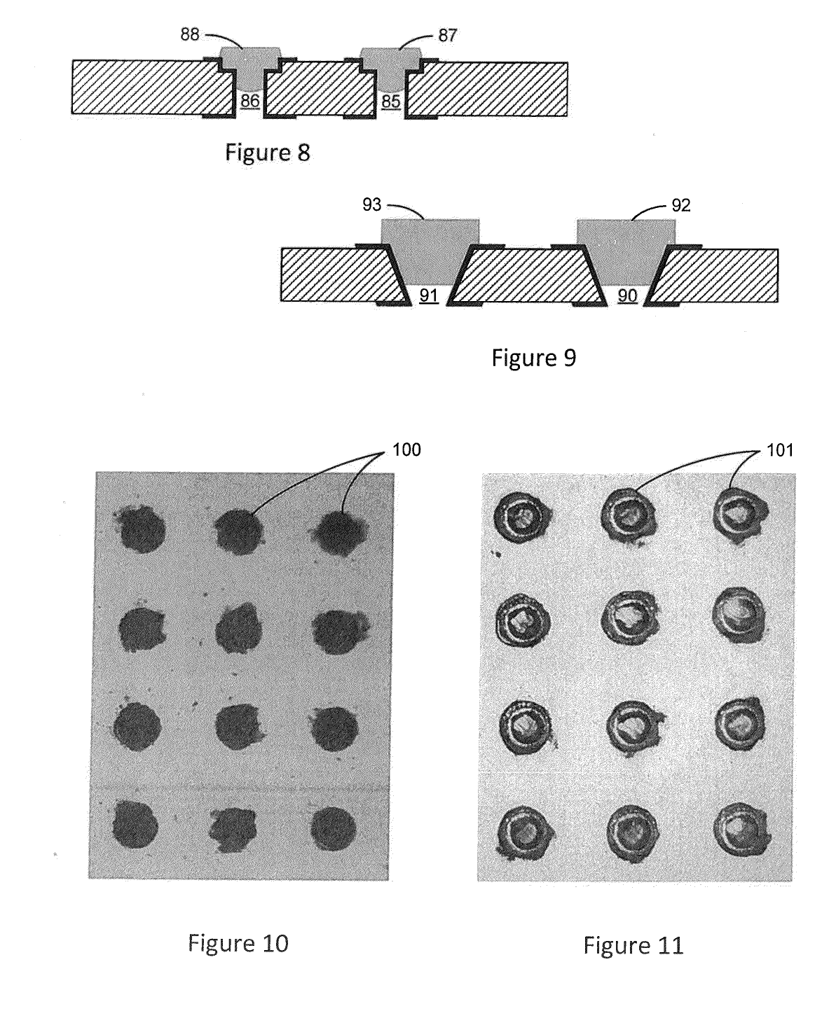

And FIGS. 8 and 9 show example alternative throughhole geometries which may be used for fabricating the extrusion-formed electrical feedthroughs of the present invention. In particular, FIG. 8 shows an example electrical feedthrough device having stepped throughholes 85 and 86 and extrusion-formed electrical feedthroughs 87 and 88, respectively, therein. And FIG. 9 shows an example electrical feedthrough device having tapered throughholes 90 and 91 and extrusion-formed electrical feedthroughs 92 and 93, respectively, therein.

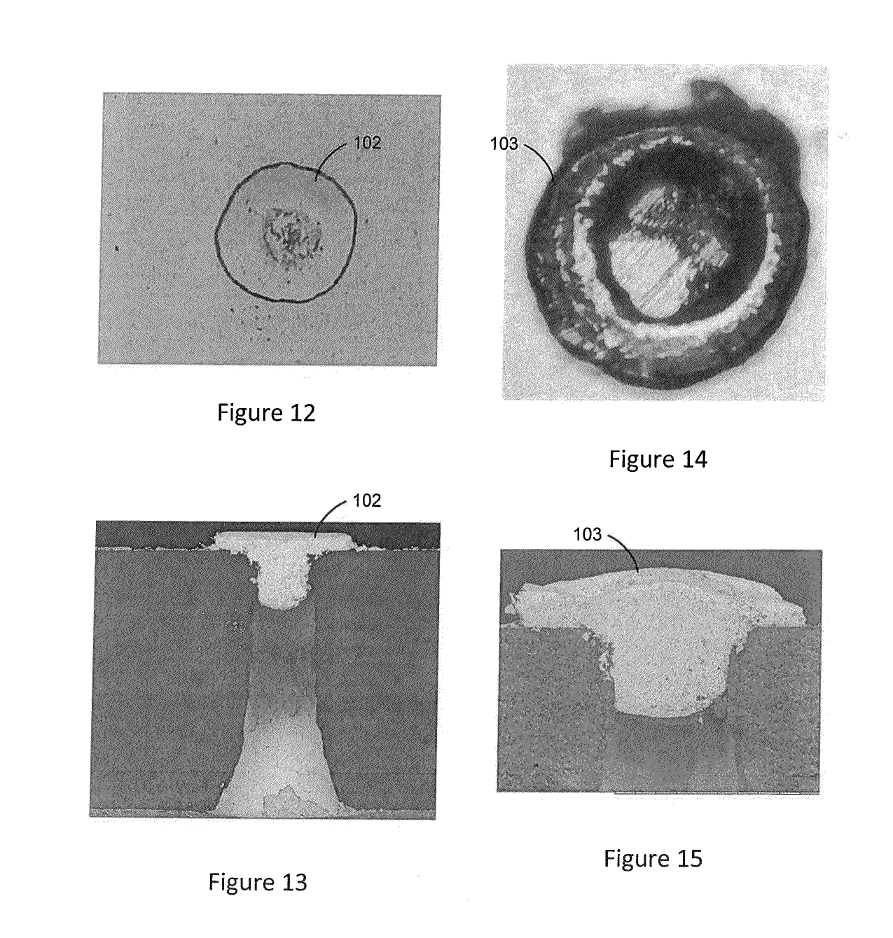

FIGS. 12 and 13 show a top-view and cross-sectional view of a single extrusion-formed hermetic electrical feedthrough 102, with the optional coining step performed. And FIGS. 14 and 15 show a top-view and cross-sectional view of a single extrusion-formed hermetic electrical feedthrough 103, without the optional coining step performed on it. As shown in FIG. 13, the coining step planarized the extruded via 102 to the alumina substrate, which is preferred for subsequent processing and assembly steps. And as can be seen in FIGS. 13 and 14, the electrical feedthroughs are shown having a rivet-like shape comprising a shank portion positioned in the throughhole, and a head portion positioned against the substrate surface at an inlet end of the throughhole

Although the description above contains many details and specifics, these should not be construed as limiting the scope of the invention or of what may be claimed, but as merely providing illustrations of some of the presently preferred embodiments of this invention. Other implementations, enhancements and variations can be made based on what is described and illustrated in this patent document. The features of the embodiments described herein may be combined in all possible combinations of methods, apparatus, modules, systems, and computer program products. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural and functional equivalents to the elements of the above-described preferred embodiment that are known to those of ordinary skill in the art are expressly incorporated herein by reference and are intended to be encompassed by the present claims. Moreover, it is not necessary for a device to address each and every problem sought to be solved by the present invention, for it to be encompassed by the present claims. Furthermore, no element or component in the present disclosure is intended to be dedicated to the public regardless of whether the element or component is explicitly recited in the claims. No claim element herein is to be construed under the provisions of 35 U.S.C. 112, sixth paragraph, unless the element is expressly recited using the phrase “means for.”

We claim:

1. A method of fabricating an electrical feedthrough structure comprising:
forming at least one throughhole(s) from one side of an electrically non-conductive substrate to an opposite side of the substrate;
conformally coating the substrate and the throughhole(s) with an electrically conductive film; and
extruding an electrically conductive material into the film-coated throughhole(s) to form extruded electrical feedthrough(s) therefrom.

2. The method of claim 1,
wherein the electrically conductive material is extruded into the film-coated throughhole(s) by stud-bumping a stud bump into each film-coated throughhole using at least one of force, elevated temperature, and ultrasonic energy.

3. The method of claim 2,
wherein the stud bumps have a larger diameter than the diameter of the film-coated throughhole(s).

4. The method of claim 1,
wherein the electrically conductive material is extruded into the film-coated throughhole(s) by: forming a corresponding number of posts made of the electrically conductive material on a sacrificial substrate, flip-chip bonding the posts in the throughholes using at least one of force, elevated temperature, and ultrasonic energy, and removing the sacrificial substrate from the flip-chip-bonded posts.

5. The method of claim 4,
wherein the posts are formed on a lift-off layer of the sacrificial substrate, and the sacrificial substrate is removed by lifting the sacrificial substrate and lift-off layer from the flip-chip bonded posts.

6. The method of claim 4,
wherein the posts have a larger diameter than the diameter of the film-coated throughhole(s).

7. The method of claim 1,
further comprising applying a compressive force to the extruded electrically conductive material into the film-coated throughhole(s).

8. The method of claim 1,
further comprising polishing at least one of the opposing sides of the substrate so that the extruded electrically conductive material is level with the polished side(s).

9. The method of claim 1,
wherein the throughhole(s) is formed with a step or taper.

10. The method of claim 1,
further comprising depositing an adhesion layer on the film-coated throughhole and substrate prior to extruding the electrically conductive material to promote adhesion between the extruded electrically conductive material and the electrically conductive film.

11. The method of claim 1,
further comprising depositing an adhesion layer on the substrate and the throughhole(s) prior to conformally coating with the electrically conductive film to promote adhesion between the electrically conductive film and the substrate.

12. An electrical feedthrough structure comprising:
an electrically non-conductive substrate having at least one throughhole(s) that is conformally coated with an electrically conductive film, and a corresponding number of electrical feedthroughs extrusion-formed in the film-coated throughhole(s) so as to be bonded to the throughhole walls.

13. The electrical feedthrough structure of claim 12,
wherein each of the electrical feedthroughs have a shank portion positioned in the throughhole, and a head portion positioned against an outer surface of the substrate at an inlet end of the throughhole.

14. The electrical feedthrough structure of claim 12,
wherein the throughhole(s) have a step or taper.

15. The electrical feedthrough structure of claim 12,
further comprising an adhesion layer between the extruded electrically conductive material and the electrically conductive film to promote adhesion therebetween.

16. The electrical feedthrough structure of claim 12,
further comprising an adhesion layer between the electrically conductive film and the substrate and the throughhole(s) to promote adhesion between therebetween.
